(19) United States
(12) Patent Application Publication
(10) Pub. No.: US 2024/0296929 A1
(43) Pub. Date: Sep. 5, 2024

(54) PRECISION COMBINATION THERAPY USING TUMOR CLONE RESPONSE PREDICTION FROM CELL DATA

(71) Applicant: International Business Machines Corporation, Armonk, NY (US)

(72) Inventors: Filippo Utro, Pleasantville, NY (US); Kahn Rhrissorrakrai, Woodside, NY (US); Laxmi Parida, Mohegan Lake, NY (US); Chaya Levovitz, New York, NY (US)

(21) Appl. No.: 18/116,114

(22) Filed: Mar. 1, 2023

(51) Int. Cl. G16H 20/30; G16B 5/00; G16B 30/00; G16B 40/20

(52) U.S. Cl. CPC G16H 20/30; G16B 5/00; G16B 30/00; G16B 40/20

(57) ABSTRACT

An AI platform is used for developing a combination therapy for a patient afflicted with a tumor that has produced clones. The combination therapy, which includes at least two perturbations, is capable of targeting clones (including subclones) that have escaped therapeutic intervention due to resistance and/or evolution. The AI platform is trained with perturbation data obtained from at least one cell line that has similar characteristics to a clone of interest. The trained AI platform predicts how the clone of interest will respond to perturbations and ranks the perturbation responses from highest to lowest. The at least one cell line may be an existing cell line from a well-established database or a synthetic cell line generated by the AI platform. The AI platform may include one or more of a machine learning platform, a deep learning platform, an artificial neural network (ANN), a convolution neural network (CNN), and a generative adversarial network (GAN).

### TECHNICAL FIELD

The present invention relates generally to prediction models, and more specifically to a prediction model that uses artificial intelligence platforms and cell line perturbation data to predict combination therapies for tumor clones.

### BACKGROUND OF THE INVENTION

Over the course of therapy, tumors typically undergo evolution in response to treatments and therapies in order to evade and/or resist elimination. The evolution of tumors is often observed by the detection of clones and subclones that have branched off from a single tumor in response to therapeutic pressure. The resistance alterations in the clones (including any subclones) enables their therapeutic escape. The implication of such tumor heterogeneity is that different clones emerging from a single tumor progenitor may harbor unique resistance mechanisms that are not responsive to traditional cancer therapies. To avoid disease progression in a patient afflicted with multiple tumor clones, a specific combination of therapies is required where each therapy within the combination is designed to target one or more of the resistance mechanisms across the different tumor clones. In view of the foregoing, there is a need in the art to develop a way to predict how an existing tumor clone may react to different therapeutic interventions.

### SUMMARY OF THE INVENTION

The present invention overcomes the need in the art by providing a prediction model that leverages perturbation data from existing cell line databases to predict the response of patient tumor clones to combination therapies comprising at least two perturbations.

In one embodiment, the present invention relates to a method comprising: sequencing a clone of interest obtained from at least one tumor lesion; identifying at least one cell line comprising characteristics similar to the clone of interest and compiling a data set comprising perturbation data for the at least one cell line; inputting the data set into an artificial intelligence (AI) platform, wherein the data set trains the AI platform to predict responses to perturbations included in the perturbation data; entering information relating to the clone of interest into the trained AI platform and obtaining as output a ranking of the predicted perturbation responses of the clone of interest to the perturbations included in the perturbation data, wherein the predicted perturbation responses are ranked from highest perturbation response to lowest perturbation response; and developing a combination therapy for the clone of interest comprising perturbations from at least two of the high-ranking perturbation responses.

In another embodiment, the present invention relates to a method comprising: sequencing a clone of interest obtained from at least one tumor lesion; identifying at least two existing cell lines comprising characteristics similar to the clone of interest and compiling a first data set comprising perturbation data for the at least two existing cell lines; inputting the first data set into an AI platform, wherein the first data set trains the AI platform to generate at least one synthetic cell line comprising perturbation data unified from the at least two existing cell lines, wherein the perturbation data for the at least one synthetic cell line is compiled into a second data set; applying the first and second data sets as training data for the AI platform to learn how to predict responses to perturbations included in the perturbation data of the first and second data sets; entering information relating to the clone of interest into the trained AI platform and obtaining as output a ranking of the predicted perturbation responses of the clone of interest to the perturbations included in the perturbation data of the first and second data sets, wherein the predicted perturbation responses are ranked from highest perturbation response to lowest perturbation response; and developing a combination therapy for the clone of interest comprising perturbations from at least two of the high-ranking perturbation responses.

In a further embodiment, the present invention relates to a computer program product for ranking tumor clone perturbation responses comprising: program instructions on one or more computer readable storage media for training an AI platform to predict responses to perturbations included in a data set comprising perturbation data for at least one cell line having characteristics similar to a clone of interest; program instructions on one or more computer readable storage media for inputting information relating to the clone of interest into the trained AI platform, wherein the trained AI platform predicts perturbation responses for the clone of interest to the perturbations included in the perturbation data of the data set; and program instructions on one or more computer readable storage media for outputting from the AI platform a ranking of the predicted perturbation responses for the clone of interest from highest perturbation response to lowest perturbation response.

In another embodiment, the present invention relates to a computer program product for ranking tumor clone perturbation responses comprising: program instructions on one or more computer readable storage media for training an AI platform to generate at least one synthetic cell line comprising perturbation data unified from perturbation data for at least two existing cell lines having characteristics similar to a clone of interest, wherein the perturbation data for the at least two existing cell lines is compiled in a first data set and the perturbation data for the at least one synthetic cell line is compiled in a second data set; program instructions on one or more computer readable storage media for training the AI platform to predict responses to the perturbations included in the perturbation data for the first and second data sets; program instructions on one or more computer readable storage media for inputting information relating to the clone of interest into the trained AI platform, wherein the trained AI platform predicts perturbation responses for the clone of interest to the perturbations included in the perturbation data of the first and second data sets; and program instructions on one or more computer readable storage media for outputting from the AI platform a ranking of the predicted perturbation responses for the clone of interest from highest perturbation response to lowest perturbation response.

In a further embodiment, the present invention relates to a system comprising: a first data set for computer input comprising perturbation data relating to at least two existing cell lines that have characteristics similar to a sequence from a clone of interest obtained from at least one tumor lesion; a second data set for computer input comprising perturbation data relating to a synthetic cell line, wherein the at least one synthetic cell line comprises perturbation data unified from the at least two existing cell lines; and an AI platform that accepts the first data set, the second data set, and information relating to the clone of interest as input and provides as output a ranking of predicted perturbation responses for the clone of interest to perturbations included in the perturbation data of the first and second data sets, wherein, the first data set trains the AI platform to generate the perturbation data for the at least one synthetic cell line, the first and second data sets train the AI platform to predict the perturbation responses of the clone of interest to the perturbations included in the perturbation data of the first and second data sets, and the predicted perturbation responses for the clone of interest are ranked from highest perturbation response to lowest perturbation response.

In another embodiment, the information relating to the clone of interest is selected from the group consisting of tumor type, tumor location, lesion location, genomics, transcriptomics, proteomics, microbiomics, metabolomics, lipidomics, epigenomics, and combinations thereof.

In another embodiment, the characteristics of the cell lines that are similar to the clone of interest are selected from the group consisting of tumor type, perturbation data, genomics, transcriptomics, proteomics, microbiomics, metabolomics, lipidomics, epigenomics, and combinations thereof.

In a further embodiment, the AI platform is selected from the group consisting of machine learning, deep learning, artificial neural networks, convolution neural networks, generative adversarial networks, and combinations thereof.

In another embodiment, the perturbations for the combination therapy are selected from the group consisting of environmental stimuli, drug inhibition, gene editing, disease treatment, and combinations thereof.

Additional embodiments and/or aspects of the invention will be provided, without limitation, in the detailed description of the invention that is set forth below.

### BRIEF DESCRIPTION OF THE DRAWINGS

FIG. 1 is a schematic diagram that illustrates the tumor clone response prediction model described herein.

FIG. 2 is a schematic diagram of a computer environment that may be used to implement the tumor clone response prediction model described herein.

### DETAILED DESCRIPTION OF THE INVENTION

Set forth below is a description of what are currently believed to be preferred aspects and/or embodiments of the claimed invention. Any alternates or modifications in function, purpose, or structure are intended to be covered by the appended claims. As used in this specification and the appended claims, the singular forms of words, including the articles “a,” “an,” and “the,” include plural referents unless the context clearly dictates otherwise. The terms “comprise,” “comprised,” “comprises,” and/or “comprising,” as used in the specification and appended claims, specify the presence of the expressly recited components, elements, features, and/or steps, but do not preclude the presence or addition of one or more other components, elements, features, and/or steps.

As used herein, the term “lesion” refers to an area of abnormal tissue and the term “tumor lesion” refers to an area of abnormal tissue that includes a complete tumor, a part of a tumor, or tumor cells.

As used herein, the term “clone” refers to a collection of cells with the same genomic profile that are considered to be homogeneous. Within the context of tumor lesions, different clonal populations that exist within a single tumor lesion are “subclones” if they share the same progenitor clone; thus, progenitor tumor clones and subclones are identical as they both represent homogenous groups of tumor cells whose therapeutic response is expected to be identical. Based on the foregoing, the term “clones of interest” as used herein, refers to progenitor tumor clones and derived subclones.

As used herein, the term “perturbation” refers to an alteration of the function of a biological system by external or internal means. Examples of perturbations include, without limitation, environmental stimuli, drug inhibition, gene editing, and disease treatment.

Examples of environmental stimuli include, without limitation, temperature changes, osmotic shock, and pressure changes.

Drug inhibition alters a biological pathway through the binding of a small molecule to an active and/or allosteric site of an enzyme. Examples of cancer growth inhibition agents include, without limitation, tyrosine kinase inhibitors, kinase protein inhibitors (e.g., mTOR and PI3K inhibitors), proteosome inhibitors, histone deacetylase inhibitors, hedgehog pathway inhibitors, BRAF inhibitors, and MEK inhibitors.

Examples of gene editing include, without limitation, gene knockout (removal or inactivation of a gene), gene knockdown (reduction in the expression of a gene), gene knockup (insertion of a protein coding cDNA sequence at a gene location), and CRISPR (clustered, regularly interspaced, short palindromic repeats that revise, remove, and replace DNA in a highly targeted manner). Within the context of the present invention, the biological system is a cell line or cells obtained from a tumor lesion, tumor clone, or tumor subclone.

Examples of disease treatment include, without limitation, cell therapy, immunotherapy, and hormone therapy. Examples of cells that may be used in cell therapy include, without limitation, autologous cells (originating from the patient), allogenic cells (originating from a donor), pluripotent stem cells (can differentiate into any cell in a living organism), multipotent stem cells (can differentiated into all cell types within a particular lineage), unipotent stem cells (can only produce one cell type, but are self-renewing), adult stem cells (responsible for maintaining and repairing the tissue in which they reside), primary cells (terminally differentiated cells isolated directly from living tissue), secondary cells (cell lines that have been immortalized and can divide indefinitely) and combinations thereof. Examples of immunotherapy include, without limitation, monoclonal antibodies, non-specific immunotherapies (e.g., cytokines and bacterial therapy), oncolytic virus therapy, T-cell therapy, and cancer vaccines. Examples of hormone therapy include, without limitation, aromatase inhibitors (AIs), estrogen receptor antagonists, selective estrogen receptor modulators (SERMs), luteinizing hormone-release hormone (LHRH), anti-androgens, CYP17 inhibitors, progestins, and adrenolytics.

As used herein, the terms “artificial intelligence,” “AI,” and “AI platform” refer to a computer algorithm that learns through experience. Examples of AI platforms include without limitation, machine learning, deep learning, and neural networks. Following is a discussion of some AI platforms that may be used to implement the present invention.

As used herein, the term “machine learning” refers to an artificial intelligence (AI) function where an algorithm learns from training data in order to make predictions or decisions without being explicitly programmed to do so. Machine learning are divided into three categories, supervised learning, semi-supervised learning, and unsupervised learning, learning. With supervised learning, a computer algorithm is trained with labeled data. In application, the computer algorithm presented with example inputs and their desired outputs, both of which are provided to the computer algorithm, and the goal of the computer algorithm is to learn a general rule that maps inputs to outputs. With unsupervised learning, a computer algorithm is trained on unlabeled data; thus, the computer algorithm is left on its own to learn through its learning algorithm how to find structure in its input. The goal of unsupervised learning is to discover hidden patterns in data through feature learning to achieve an end goal. With semi-supervised learning, a computer algorithm is trained on a small amount of labeled data and a large amount of unlabeled data.

As used herein, the term “deep learning” refers to an AI function that mimics the workings of the human brain in processing and categorizing data. Deep learning-based AI is able to learn from data that is unstructured and unlabeled. In operation, deep learning-based AI programs find correlations between inputs and outputs by learning to approximate an unknown function (f(x)=y) between any input x and any output y, assuming they are related by correlation or causation.

As used herein, the term “artificial neural network” or “ANN” refers to a deep learning AI function that is modeled after the human brain with a collection of simulated neurons, which are all fully connected. Each neuron is a node that is connected to other nodes via links that are analogous to biological axo-synapse dendrite connections. Each link has a weight, which determines the strength of one node's influence on another. Neural networks learn (i.e., are trained) by processing examples, each of which contains a known input and output forming probability weighted associations between the input and the output. In operation, a neural network groups unlabeled input data according to similarities among example inputs, automatically extracts features from the groups, clusters groups with similar features, and classifies output data when there is a labeled dataset for training. The patterns recognized by a neural network are numerical and contained in vectors, which must be translated. Examples of neural network vectors include without limitation, images, sound, text, time, or combinations thereof.

As used herein, the term “convolutional neural network” or “CNN” refers to a neural network that uses convolutional layers to convolve input and pass its result to the next layer. While fully connected feedforward neural networks can be used to learn features and classify data, CNNs regularize multilayer fully connected networks where each neuron in one layer is connected to all neurons on the next layer. The nature of the fully connected layers of a CNN leads to overfitting data, which must be regularized. CNNs regularize by taking advantage of the hierarchical pattern in data to assemble complex patterns using smaller and simpler patterns. A CNN consists of an input layer, hidden middle layers, and an output layer. The hidden middle layers perform the convolutions. After passing through a convolutional layer, the data become abstracted to a feature map, which is the output of the convolution kernel that was applied to the previous layer.

As used herein, the terms “GAN” (generative adversarial network) and “GAN model” refer to a machine learning function with two neural networks, a generator and adversary (also referred to herein as a discriminator) that enter into a contest with each other in the form of a zero-sum game where one of the neural network's gains is the other neural network's loss. GAN is based on indirect training of the generator through the discriminator where the generator generates candidates that the discriminator evaluates. For the evaluation, the discriminator instructs the generator on how realistic the generator input seems. The generator's training objective is to increase the error rate of the discriminator by producing novel candidates that the discriminator thinks are not synthesized (i.e., are part of the true data distribution). A known data set serves as the initial training data for the discriminator where the training involves presenting the discriminator with samples from the training dataset until the discriminator achieves acceptable accuracy. The generator is trained based upon whether it succeeds in fooling the discriminator into thinking that the synthetic data is the existing data. A GAN generator network is typically seeded with randomized input that is sampled from a predefined latent space e.g., a multivariate normal distribution. Thereafter, candidates synthesized by the generator are evaluated by the discriminator. The machine learning function for a GAN can be of any of the three categories, specifically, fully supervised, semi-supervised, or unsupervised machine learning. The two neural networks of the GAN may be two ANNs, two CNNs, or a combination of one ANN and one CNN.

The present invention trains an AI platform to predict how a tumor clone will behave in response to different perturbations by training the AI platform on cell line perturbation data. Because a tumor typically has multiple clones and a patient may have multiple tumors, the prediction response at the clonal level will generally require combination therapy comprising at least two perturbations in order to target the behavior of the multiple clones as they evolve from their tumor progenitor. To the best of the inventor's knowledge, the present invention is the first tool to predict tumor clone perturbation responses.

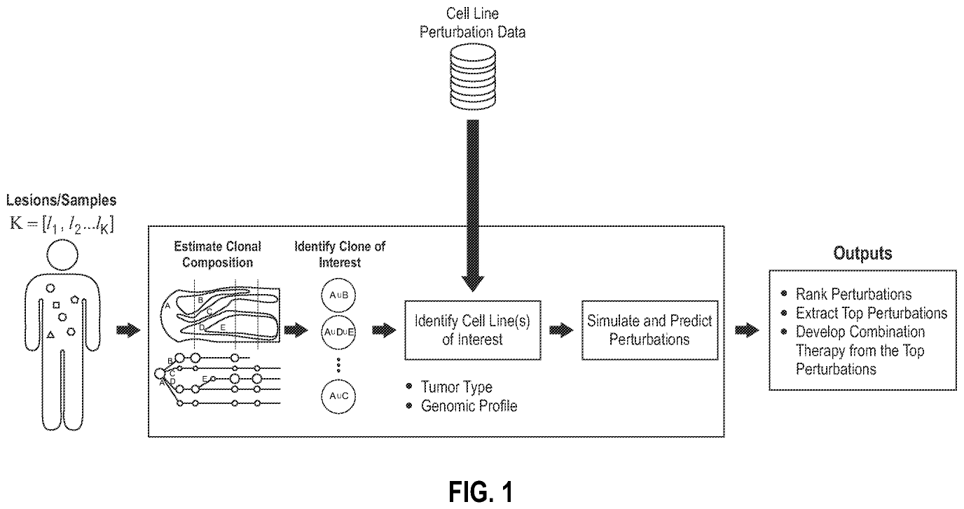

FIG. 1 is a schematic representation of the workflow required to implement the prediction model described herein. As a starting point, at least one tumor lesion (K=l₁, l₂, . . . lₖ) is taken from a single patient and sequenced in order to determine the clonal composition of the at least one tumor lesion, including progenitor tumor clones and subclones derived from the progenitor tumor clones. From the sequencing data, clones of interest are identified based upon selected characteristics, including, without limitation, tumor type (e.g., the type of cancer associated with the tumor, such as bladder cancer tumor, breast cancer tumor, colorectal cancer tumor, kidney cancer tumor, lung cancer tumor, etc.), tumor location, lesion location, and omic data. Examples of omic data include, without limitation, genomics (information relating to genes), transcriptomics (information relating to RNA), proteomics (information relating to proteins), microbiomics (information relating to microorganisms, such as bacteria, fungi, and viruses), metabolomics (information relating to metabolites), lipidomics (information relating to lipids) and epigenomics (information relating to methylated DNA or modified histone proteins). Next, one or more cell lines from one or more established databases that share the characteristics with a clone of interest are identified. It is to be understood that one or more cell lines may share characteristics with a single clone of interest. Examples of databases that can be used to identify the at least one cell line includes, without limitation, the Sanger databases (available at https://cancer.sanger.ac.uk/cell_lines), the DepMap Portal (available at https://depmap.org/portal/), the CCLE (Cancer Cell Line Encyclopedia available at https://sites.broadinstitute.org/ccle/), the Library of Network-based Cellular Signatures (LINCS) (available at https://lincsproject.org/), and combinations thereof.

The Sanger databases include COSMIC (Catalogue of Somatic Mutations in Cancer, which is an expert-curated database of somatic mutations), Cell Lines Project (mutation profiles of over 1000 cell lines used in cancer research), COSMIC-3D (contains an interactive view of cancer mutations in the context of 3-D structures), Cancer Gene Census (a catalogue of genes with mutations that are causally implicated in cancer), Cancer Mutation Census (a classification of genetic variants driving cancer), and Actionability (mutations actionable in precision oncology).

The DepMap portal provides a cancer dependency map that incudes genetic and pharmacological dependencies, tumor contexts, predictive biomarkers, and over 2000 cancer models.

The CCLE includes cell line annotations for over 1000 human cancer models, merged mutation calls for 329 cell lines, RNA expression data for 1019 cell lines, fusion calls for 1019 cell lines, epigenetic and histone modification data, proteomic data, and metabolomic data. In addition to the foregoing, any private cell line database (such as those owned by biotechnology or pharmaceutical companies). In one embodiment, the any one of the foregoing databases may be used to identify at least one cell line similar to the clone of interest by comparing the genomic profile of the clone of interest to genetic profiles for the at least one cell line.

The LINCS database identifies and categorizes molecular signatures that occur when cells are exposed to agents that perturb their normal function.

In some situations, it is possible that there may be few cell lines in the known databases (also referred to herein as “existing cell lines”) that closely match a clone of interest. Such situations may include a type of cancer that has a high diversity of tumor clones across all cancer patients or clones that are unique. In such situations, it becomes statistically difficult to identify recurrent features and patterns to identify existing cell lines of interest from which to make predictions. In order to identify shared mechanisms between clones and to identify perturbations that may target multiple clones, existing cell lines that are similar to two or more clones of interest may be unified into one or more synthetic cell lines. The one or more synthetic cell lines allow for the generation of predictions on the tumor lesion as a whole (since a tumor lesion is an embodiment of one or more clones of interest). It is to be understood that depending on the type of tumor lesions that generate the clones of interest, the cell lines used in the prediction model described herein may be existing cell lines and/or synthetic cell lines.

Once the one or more cell lines are selected from the existing databases, information relating to the one or more cell lines is compiled in a tabular format for input into an AI platform as training data. Examples of information relating to the one or more cell lines includes, without limitation, tumor type, perturbation data, and omic profiles. Examples of perturbation data includes, without limitation, the response of the one or more cell lines to different environmental stimuli, drug inhibition, gene editing, and disease treatment. Examples of omic profiles include, without limitation, genomics, transcriptomics, proteomics, microbiomics, metabolomics, lipidomics, and epigenomics. Examples of tabular data formats that may be used for the input include, without limitation, comma separated value (CSV) files and spreadsheets (e.g., EXCEL®, Microsoft Corporation, Redmond, WA, USA; NUMBERS®, Apple Inc., Cupertino, CA, USA)).

To generate a synthetic cell line, the information relating to at least two existing cell lines is prepared into the tabular format and input into the AI platform with instructions for the AI platform to unify the information from the at least two cell lines into one or more synthetic cell lines. In one embodiment, the AI platform can be a GAN that takes as input these two or more cell lines to generate one or more synthetic cell lines with omic features similar to the original input.

The AI platform is trained on the cell line perturbation data in the tabular format in order to learn the responses of the one or more cell lines (both existing and synthetic) to the perturbations. The training on the cell line perturbation data allows the AI platform to predict how the clones of interest (which shares similar characteristics to the cell lines) will respond to different perturbations. Any AI platform may be used to generate the clone of interest perturbation responses, including, without limitation, a machine learning platform, a deep learning platform, a neural network, a CNN, a GAN, and combinations thereof.

Once the predicted perturbation responses are obtained for a clone of interest, the perturbation responses are ranked from the greatest perturbation response to the least perturbation response. The perturbation response rankings are taken together to develop a combination therapy designed to treat the clone of interest. In one embodiment, for any one clone of interest, the combination therapy will generally include the highest-ranking perturbations from two or more of the perturbations described herein. In another embodiment, where appropriate, drug synergy and/or toxicity is analyzed to ensure that the combination therapy avoids harmful side effects or drug interactions. By providing a combination therapy that is designed to safely target all clones in a patient, the risk of disease progression for the patient may be greatly reduced.

By way of example, for a patient with multiple tumor lesions that have each produced their own clones, the existence of the different heterogeneous clones within the single patient may require different combinations in order to target the different resistance mechanisms of the heterogeneous clones. For example, for any two heterogenous clones that are tested with the prediction model described herein, one clone may be predicted to respond to combination therapy with the highest ranking gene editing perturbation of knock-out gene editing and the highest ranking disease treatment perturbation of pluripotent stem cell treatment while another clone may be predicted to respond to combination therapy with the highest ranking environmental perturbation of osmotic shock, the highest ranking drug inhibition perturbation tyrosine kinase drug inhibition, and the highest ranking disease treatment perturbation of monoclonal antibody therapy.

In one embodiment, the present invention comprises sequencing a clone of interest obtained from at least one tumor lesion; identifying at least one cell line comprising characteristics similar to the clone of interest and compiling a data set comprising perturbation data for the at least one cell line; inputting the data set into an AI platform, wherein the data set trains the AI platform to predict responses to perturbations included in the perturbation data; entering information relating to the clone of interest into the trained AI platform and obtaining as output a ranking of the predicted perturbation responses of the clone of interest to the perturbations included in the perturbation data, wherein the predicted perturbation responses are ranked from highest perturbation response to lowest perturbation response; and developing a combination therapy for the clone of interest comprising perturbations from at least two of the high-ranking perturbation responses.

In another embodiment, the present invention comprises sequencing a clone of interest obtained from at least one tumor lesion; identifying at least two existing cell lines comprising characteristics similar to the clone of interest and compiling a first data set comprising perturbation data for the at least two existing cell lines; inputting the first data set into an artificial intelligence (AI) platform, wherein the first data set trains the AI platform to generate at least one synthetic cell line comprising perturbation data unified from the at least two existing cell lines, wherein the perturbation data for the at least one synthetic cell line is compiled into a second data set; applying the first and second data sets as training data for the AI platform to learn how to predict responses to perturbations included in the perturbation data of the first and second data sets; entering information relating to the clone of interest into the trained AI platform and obtaining as output a ranking of the predicted perturbation responses of the clone of interest to the perturbations included in the perturbation data of the first and second data sets, wherein the predicted perturbation responses are ranked from highest perturbation response to lowest perturbation response; and developing a combination therapy for the clone of interest comprising perturbations from at least two of the high-ranking perturbation responses.

In a further embodiment, the present invention comprises program instructions on one or more computer readable storage media for training an AI platform to predict responses to perturbations included in a data set comprising perturbation data for at least one cell line having characteristics similar to a clone of interest; program instructions on one or more computer readable storage media for inputting information relating to the clone of interest into the trained AI platform, wherein the trained AI platform predicts perturbation responses for the clone of interest to the perturbations included in the perturbation data of the data set; and program instructions on one or more computer readable storage media for outputting from the AI platform a ranking of the predicted perturbation responses for the clone of interest from highest perturbation response to lowest perturbation response.

In another embodiment, the present invention comprises program instructions on one or more computer readable storage media for training an AI platform to generate at least one synthetic cell line comprising perturbation data unified from perturbation data for at least two existing cell lines having characteristics similar to a clone of interest, wherein the perturbation data for the at least two existing cell lines is compiled in a first data set and the perturbation data for the at least one synthetic cell line is compiled in a second data set; program instructions on one or more computer readable storage media for training the AI platform to predict responses to the perturbations included in the first and second data sets; program instructions on one or more computer readable storage media for inputting information relating to the clone of interest into the trained AI platform, wherein the trained AI platform predicts perturbation responses for the clone of interest to the perturbations included in the first and second data sets; and program instructions on one or more computer readable storage media for outputting from the AI platform a ranking of the predicted perturbation responses for the clone of interest from highest perturbation response to lowest perturbation response.

In a further embodiment, the present invention comprises a first data set for computer input comprising perturbation data relating to at least two existing cell lines that have characteristics similar to a sequence from a clone of interest obtained from at least one tumor lesion; a second data set for computer input comprising perturbation data relating to a synthetic cell line, wherein the at least one synthetic cell line comprises perturbation data unified from the at least two existing cell lines; and an AI platform that accepts the first data set, the second data set, and information relating to the clone of interest as input and provides as output a ranking of predicted perturbation responses for the clone of interest to perturbations included in the perturbation data of the first and second data sets, wherein, the first data set trains the AI platform to generate the perturbation data for the at least one synthetic cell line, the first and second data sets train the AI platform to predict the perturbation responses of the clone of interest to the perturbations included in the perturbation data of the first and second data sets, and the predicted perturbation responses for the clone of interest are ranked from highest perturbation response to lowest perturbation response.

In another embodiment, the information relating to the clone of interest is selected from tumor type, tumor location, lesion location, omic profiles, and combinations thereof.

In a further embodiment, the characteristics of the existing cell line that are similar to the clone of interest is selected from tumor type, perturbation data, omic profiles, and combinations thereof.

In another embodiment, the perturbations for the combination therapy are selected from the group consisting of environmental stimuli, drug inhibition, gene editing, disease treatment, and combinations thereof.

In a further embodiment, the AI platform is selected from the group consisting of machine learning, deep learning, ANNs, CNNs, GANs, and combinations thereof.

In another embodiment, the AI platform comprises a GAN, alone or in combination with an ANN or a CNN.

The present invention has applications for personalized medicine and patient screenings. For personalized medicine, the prediction model described herein has utility in developing treatments that are specific to an individual that has at least one type of tumor. For patient screening, the prediction model may be used to screen patients for clinical trials based upon the clonal composition of the patient's tumor lesions.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random-access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

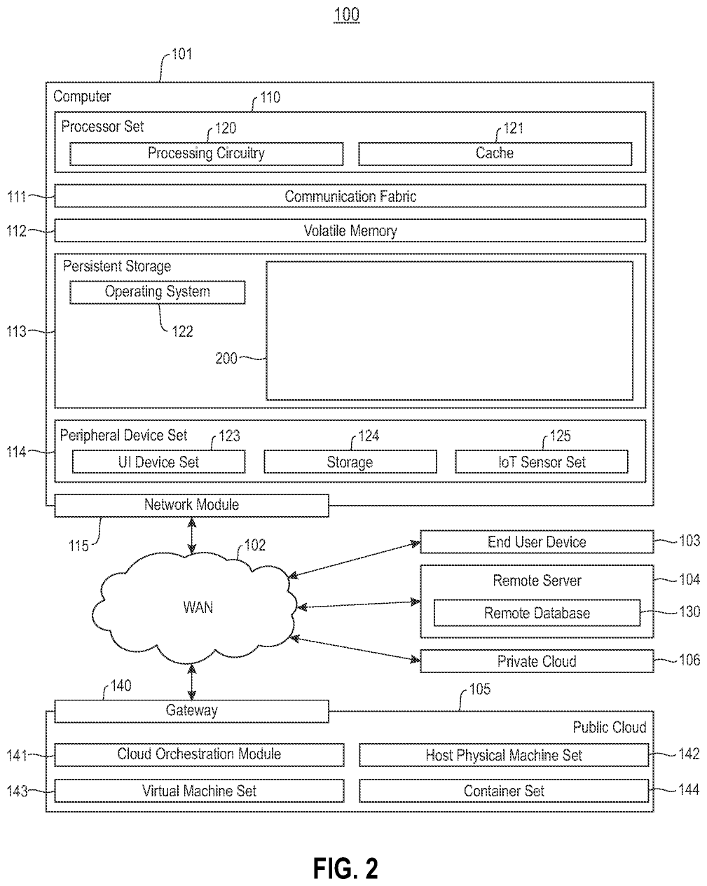

The following discussion refers to FIG. 2. Computing environment 100 contains an example of an environment for the execution of at least some of the computer code involved in performing the inventive methods, such as the computer code required for running machine learning, deep learning, ANNs, CNNs, and GANs to predict subclone perturbation responses as described herein 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 2. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various aspects and/or embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the aspects and/or embodiments, the practical application or technical improvement over technologies found in the marketplace, or to enable others of ordinary skill in the art to understand the aspects and/or embodiments disclosed herein.

### EXPERIMENTAL

The following examples are set forth to provide those of ordinary skill in the art with a complete disclosure of how to make and use the aspects and embodiments of the invention as set forth herein. While efforts have been made to ensure accuracy with respect to variables, experimental error and deviations should be considered.

### Example 1

Four tumor lesions are removed from the liver (2 samples), brain (1 sample), and subcutaneous soft tissue (1 sample) of a patient afflicted with metastatic colon cancer. Cells from the four tumor lesions are sequenced. Clonal analysis is performed on the cells of the four tumor lesions using the open source software program Concerti (available at https://github.com/ComputationalGenomics/Concerti) and two sibling clones are identified: one clone with a KRAS p.G12S allele and the other clone with an ELF3 p.S229R allele. The clone with the KRAS p.G12S allele develops two subclones, both of which have the BCLAF p.S496L allele and are specific to liver tissue. The clone with the ELF3 p.S229R allele also develops two subclones, both of which maintain the ELF3 p.S229R allele, but with one specific to brain tissue and the other specific to subcutaneous soft tissue. All four clones are identified as clones of interest.

For each of the four clones of interest, the clone's tumor type, tumor location, lesion location, genomic profile, and any other omic profiles are used as to identify ten cell lines from within the Sanger, DepMap, CCLE, and LINCS databases as bearing similarities to the subclones of interest. Information obtained from the cell line databases are obtained as a csv file. The cell line information includes tumor type, perturbation data, genomic profile, and any other omic profiles. The csv file of all ten cell lines is applied as input into a GAN model to generate synthetic cell line data of similar genomic profiles to the identified cell lines to supplement and expand the perturbation dataset. The similarity between the clone of interest and the synthetic and existing cell lines is measured for a more accurate assessment of the fit of these cell lines and their perturbation data to the clone of interest. A machine learning model such as an ANN is then used to predict perturbation response by training on the existing and synthesized data. Once the ANN is trained, the tumor type, tumor location, lesion location, genomic profile, and any other omic profiles of the clones of interest are provided to the ANN and the output from the ANN is a ranking of predicted perturbation responses for the clone of interest. This process is repeated for each clone of interest to treat them individually.

The GAN model is built using the open-source deep learning frameworks PyTorch, Tensorflow, and/or KERAS, all of which use Python computer programming language and allow for the building of the GAN with a training function as well an ANN and/or CNN generator and discriminator. The GAN may be built with an ANN generator and an ANN discriminator, a CNN generator and a CNN discriminator, an ANN generator and a CNN discriminator, or an CNN generator and an ANN discriminator. Once built, the GAN is implemented using supervised, unsupervised, or semi-supervised machine learning.

### Example 2

Five tumor lesions are removed from the liver (2 samples) and kidney (3 sample) of a patient afflicted with metastatic breast cancer. Cells from the five tumor lesions are sequenced. Clonal analysis is performed on the cells of the five tumor lesions using Concerti and a total of twelve clones of interest are found from across the five tumor lesions. Each of the twelve clones of interest are found to harbor some shared alterations, but are primarily genetically distinct. For all twelve of the clones of interest, the clone's tumor type, tumor location, lesion location, genomics and other omic profiles are used to identify thirty cell lines from within the Sanger, DepMap, CCLE, and LINCS databases as bearing similarities to the clones of interest. Information obtained from the cell line databases are obtained as a csv file. The cell line information includes tumor type, perturbation data, genomic profile, and any other omic profiles. The csv file of all thirty cell lines is applied as input into a GAN model to generate synthetic cell line data of similar genomic profiles to the identified cell lines to supplement and expand the perturbation dataset. The similarity between the clones of interest and the synthetic and existing cell lines is measured for a more accurate assessment of the fit of these cell lines and their perturbation data to the clone of interest. A machine learning model such as an ANN is then used to predict perturbation response by training on the existing and synthesized data. Once the ANN is trained, the tumor type, tumor location, lesion location, genomic profile, and any other omic profiles of the clones of interest are provided to the ANN and the output from the ANN is a ranking of predicted perturbation responses for the clones of interest. This process is repeated for each clone of interest to treat them individually.

The GAN model is built using the open-source deep learning frameworks PyTorch, Tensorflow, and/or KERAS, all of which use Python computer programming language and allow for the building of the GAN with a training function as well an ANN and/or CNN generator and discriminator. The GAN may be built with an ANN generator and an ANN discriminator, a CNN generator and a CNN discriminator, an ANN generator and a CNN discriminator, or an CNN generator and an ANN discriminator. Once built, the GAN is implemented using supervised, unsupervised, or semi-supervised machine learning.

We claim:

1. A method comprising:
sequencing a clone of interest obtained from at least one tumor lesion;
identifying at least one cell line comprising characteristics similar to the clone of interest and compiling a data set comprising perturbation data for the at least one cell line;
inputting the data set into an artificial intelligence (AI) platform, wherein the data set trains the AI platform to predict responses to perturbations included in the perturbation data;
entering information relating to the clone of interest into the trained AI platform and obtaining as output a ranking of the predicted perturbation responses of the clone of interest to the perturbations included in the perturbation data, wherein the predicted perturbation responses are ranked from highest perturbation response to lowest perturbation response; and
developing a combination therapy for the clone of interest comprising perturbations from at least two of the high-ranking perturbation responses.

2. The method of claim 1, wherein the characteristics of the at least one cell line that are similar to the clone of interest are selected from the group consisting of tumor type, perturbation data, genomics, transcriptomics, proteomics, microbiomics, metabolomics, lipidomics, epigenomics, and combinations thereof.

3. The method of claim 1, wherein the AI platform is selected from the group consisting of machine learning, deep learning, artificial neural networks, convolution neural networks, generative adversarial networks, and combinations thereof.

4. The method of claim 1, wherein the information relating to the clone of interest is selected from the group consisting of tumor type, tumor location, lesion location, genomics, transcriptomics, proteomics, microbiomics, metabolomics, lipidomics, epigenomics, and combinations thereof.

5. The method of claim 1, wherein the perturbations for the combination therapy are selected from the group consisting of environmental stimuli, drug inhibition, gene editing, disease treatment, and combinations thereof.

6. A method comprising:
sequencing a clone of interest obtained from at least one tumor lesion;
identifying at least two existing cell lines comprising characteristics similar to the clone of interest and compiling a first data set comprising perturbation data for the at least two existing cell lines;
inputting the first data set into an artificial intelligence (AI) platform, wherein the first data set trains the AI platform to generate at least one synthetic cell line comprising perturbation data unified from the at least two existing cell lines, wherein the perturbation data for the at least one synthetic cell line is compiled into a second data set;
applying the first and second data sets as training data for the AI platform to learn how to predict responses to perturbations included in the perturbation data of the first and second data sets;
entering information relating to the clone of interest into the trained AI platform and obtaining as output a ranking of the predicted perturbation responses of the clone of interest to the perturbations included in the perturbation data of the first and second data sets, wherein the predicted perturbation responses are ranked from highest perturbation response to lowest perturbation response; and
developing a combination therapy for the clone of interest comprising perturbations from at least two of the high-ranking perturbation responses.

7. The method of claim 6, wherein the characteristics of the at least two existing cell lines that are similar to the clone of interest are selected from the group consisting of tumor type, perturbation data, genomics, transcriptomics, proteomics, microbiomics, metabolomics, lipidomics, epigenomics, and combinations thereof.

8. The method of claim 6, wherein the AI platform is selected from the group consisting of machine learning, deep learning, artificial neural networks, convolution neural networks, generative adversarial networks, and combinations thereof.

9. The method of claim 6, wherein the information relating to the clone of interest is selected from the group consisting of tumor type, tumor location, lesion location, genomics, transcriptomics, proteomics, microbiomics, metabolomics, lipidomics, epigenomics, and combinations thereof.

10. The method of claim 6, wherein the perturbations for the combination therapy are selected from the group consisting of environmental stimuli, drug inhibition, gene editing, disease treatment, and combinations thereof.

11. A computer program product for ranking tumor clone perturbation responses comprising:
program instructions on one or more computer readable storage media for training an artificial intelligence (AI) platform to predict responses to perturbations included in a data set comprising perturbation data for at least one cell line having characteristics similar to a clone of interest;
program instructions on one or more computer readable storage media for inputting information relating to the clone of interest into the trained AI platform, wherein the trained AI platform predicts perturbation responses for the clone of interest to the perturbations included in the perturbation data of the data set; and
program instructions on one or more computer readable storage media for outputting from the AI platform a ranking of the predicted perturbation responses for the clone of interest from highest perturbation response to lowest perturbation response.

12. The computer program product of claim 11, wherein the AI platform is selected from the group consisting of machine learning, deep learning, artificial neural networks, convolution neural networks, generative adversarial networks, and combinations thereof.

13. The computer program product of claim 11, wherein the AI platform comprises a generative adversarial network.

14. The computer program product of claim 11, wherein the information relating to the clone of interest is selected from the group consisting of tumor type, tumor location, lesion location, genomics, transcriptomics, proteomics, microbiomics, metabolomics, lipidomics, epigenomics, and combinations thereof.

15. The computer program product of claim 11, wherein the perturbations included in the data set are selected from the group consisting of environmental stimuli, drug inhibition, gene editing, disease treatment, and combinations thereof.

16. A computer program product for ranking tumor clone perturbation responses comprising:
program instructions on one or more computer readable storage media for training an artificial intelligence (AI) platform to generate at least one synthetic cell line comprising perturbation data unified from perturbation data for at least two existing cell lines having characteristics similar to a clone of interest, wherein the perturbation data for the at least two existing cell lines is compiled in a first data set and the perturbation data for the at least one synthetic cell line is compiled in a second data set;
program instructions on one or more computer readable storage media for training the AI platform to predict responses to the perturbations included in the perturbation data for the first and second data sets;
program instructions on one or more computer readable storage media for inputting information relating to the clone of interest into the trained AI platform, wherein the trained AI platform predicts perturbation responses for the clone of interest to the perturbations included in the perturbation data of the first and second data sets; and
program instructions on one or more computer readable storage media for outputting from the AI platform a ranking of the predicted perturbation responses for the clone of interest from highest perturbation response to lowest perturbation response.

17. The computer program product of claim 16, wherein the AI platform is selected from the group consisting of machine learning, deep learning, artificial neural networks, convolution neural networks, generative adversarial networks, and combinations thereof.

18. The computer program product of claim 16, wherein the AI platform comprises a generative adversarial network.

19. The computer program product of claim 16, wherein the information relating to the clone of interest is selected from the group consisting of tumor type, tumor location, lesion location, genomics, transcriptomics, proteomics, microbiomics, metabolomics, lipidomics, epigenomics, and combinations thereof.

20. The computer program product of claim 16, wherein the perturbations included in the first and second data are selected from the group consisting of environmental stimuli, drug inhibition, gene editing, disease treatment, and combinations thereof.

21. A system comprising:
a first data set for computer input comprising perturbation data relating to at least two existing cell lines that have characteristics similar to a sequence from a clone of interest obtained from at least one tumor lesion;
a second data set for computer input comprising perturbation data relating to a synthetic cell line, wherein the at least one synthetic cell line comprises perturbation data unified from the at least two existing cell lines; and
an artificial intelligence (AI) platform that accepts the first data set, the second data set, and information relating to the clone of interest as input and provides as output a ranking of predicted perturbation responses for the clone of interest to perturbations included in the perturbation data of the first and second data sets, wherein,
the first data set trains the AI platform to generate the perturbation data for the at least one synthetic cell line,
the first and second data sets train the AI platform to predict the perturbation responses of the clone of interest to the perturbations included in the perturbation data of the first and second data sets, and
the predicted perturbation responses for the clone of interest are ranked from highest perturbation response to lowest perturbation response.

22. The system of claim 21, wherein the AI platform is selected from the group consisting of machine learning, deep learning, artificial neural networks, convolution neural networks, generative adversarial networks, and combinations thereof.

23. The system of claim 21, wherein the AI platform comprises a generative adversarial network.

24. The system of claim 21, wherein the information relating to the clone of interest is selected from the group consisting of tumor type, tumor location, lesion location, genomics, transcriptomics, proteomics, microbiomics, metabolomics, lipidomics, epigenomics, and combinations thereof.

25. The system of claim 21, wherein the perturbations included in the first and second data sets are selected from the group consisting of environmental stimuli, drug inhibition, gene editing, disease treatment, and combinations thereof.
